(19) United States
(12) Patent Application Publication
(10) Pub. No.: US 2022/0348662 A1
(43) Pub. Date: Nov. 3, 2022

(54) USE OF FGFR INHIBITORS IN FGFR-GENETICALLY ALTERED CANCERS TO ENHANCE PATIENT RESPONSE TO IMMUNE CHECKPOINT INHIBITORS IN SEQUENTIAL TREATMENT SETTINGS

(71) Applicant: JANSSEN PHARMACEUTICA NV, Beerse (BE)

(72) Inventors: Waleed S. SHALABY, Horsham, PA (US); Ademi Elena SANTIAGO-WALKER, Spring House, PA (US); Raluca VERONA, Spring House, PA (US); Enrique ZUDAIRE, Spring House, PA (US)

(21) Appl. No.: 17/763,251

(22) PCT Filed: Sep. 25, 2020
(86) PCT No.: PCT/EP2020/076999

§ 371 Date: Mar. 24, 2022

Related U.S. Application Data
(60) Provisional application No. 62/906,517, filed on Sep. 26, 2019.

(51) Int. Cl. C07K 16/28; A61K 31/498; A61K 39/395; A61P 35/04; A61P 13/02

(52) U.S. Cl. CPC C07K 16/2818; C07K 16/2827; A61K 31/498; A61K 39/39558; A61P 35/04; A61P 13/02; A61K 2039/505

(57) ABSTRACT

Embodiments of the present invention relate to a method of treating cancer in a patient comprising administering an immune checkpoint inhibitor to the patient, wherein the patient has been diagnosed with an FGFR-genetically altered cancer, and has been pre-treated with an FGFR inhibitor, such as erdafitinib.

### CROSS-REFERENCE TO RELATED APPLICATIONS

This application claims the benefit of priority of U.S. Provisional Application No. 62/906,517, filed on Sep. 26, 2019, which is incorporated by reference herein, in its entirety and for all purposes.

### FIELD OF THE INVENTION

The present invention relates to methods of treating cancers using fibroblast growth factor receptor (FGFR) inhibitors. In particular, the present invention relates to methods of using FGFR inhibitors in FGFR-genetically altered cancers to enhance patient response to immune checkpoint inhibitors in sequential treatment settings.

### BACKGROUND

Urothelial carcinoma (UC) is the most common form of bladder cancers and nearly 20% of patients with metastatic UC (mUC) have fibroblast growth factor receptor (FGFR) gene alterations. Clinical outcomes with platinum-based or taxane chemotherapy and immunotherapy (checkpoint inhibitors) have been suboptimal and there exists a significant unmet treatment need for mUC. It is accordingly an object of the present disclosure to provide such methods.

### SUMMARY

According to particular embodiments, the present invention relates to methods of treating any FGFR-genetically altered cancer with sequential systemic or local therapies, wherein the patient is first administered an FGFR inhibitor for a period of time, which functions to “prime” the immune system, and the patient is subsequently administered an immune checkpoint inhibitor for a period of time, e.g., until progression of disease. According to certain embodiments, the patient's response to the immune checkpoint inhibitor following administration of the FGFR inhibitor is greater than the patient's response to an immune checkpoint inhibitor in the absence of pre-treatment with an FGFR inhibitor.

According to particular embodiments, the present invention provides a method of treating cancer in a patient comprising administering a therapeutically effective amount of an immune checkpoint inhibitor to the patient, wherein the patient has an FGFR genetic variant (in particular a FGFR mutation or FGFR fusion), and has been treated with an FGFR inhibitor. Stated another way, the method may comprise administering an FGFR inhibitor to the patient for a period of time (e.g., as a monotherapy and/or without simultaneous administration of an immune checkpoint inhibitor), and after said period of time, administering an immune checkpoint inhibitor to the patient for a subsequent period of time (e.g., as a monotherapy and/or without simultaneous administration of an FGFR inhibitor). According to particular embodiments, the present invention provides an immune checkpoint inhibitor for use in the treatment of cancer in a patient, wherein the patient has an FGFR genetic variant (in particular a FGFR mutation or FGFR fusion), and has been treated with an FGFR inhibitor. Stated another way, the present invention provides an FGFR inhibitor for use in the treatment of cancer in a patient for a period of time (e.g., as a monotherapy and/or without simultaneous administration of an immune checkpoint inhibitor), and after said period of time, administering an immune checkpoint inhibitor to the patient for a subsequent period of time (e.g., as a monotherapy and/or without simultaneous administration of an FGFR inhibitor). According to particular embodiments, the present invention provides the use of an immune checkpoint inhibitor for the manufacture of a medicament for the treatment of cancer in a patient, wherein the patient has an FGFR genetic variant (in particular a FGFR mutation or FGFR fusion), and has been treated with an FGFR inhibitor. Stated another way, the present invention provides the use of an FGFR inhibitor for the manufacture of a medicament for the treatment of cancer in a patient for a period of time (e.g., as a monotherapy and/or without simultaneous administration of an immune checkpoint inhibitor), and after said period of time, administering an immune checkpoint inhibitor to the patient for a subsequent period of time (e.g., as a monotherapy and/or without simultaneous administration of an FGFR inhibitor).

As used herein, administration of a drug for a period of time refers to a particular number of days, weeks, or months during which time the patient is administered the drug according to a prescribed dosing regimen for said drug (e.g., daily, twice daily, etc.). According to particular embodiments, when an FGFR inhibitor is administered for a period of time, an immune checkpoint inhibitor is not administered during that period of time. Likewise, according to particular embodiments, when an immune checkpoint inhibitor is administered for a period of time, an FGFR inhibitor is not administered during that period of time.

According to particular embodiments, the FGFR inhibitor is erdafitinib or a pharmaceutically acceptable salt thereof.

According to particular embodiments, prior to the step of administering the immune checkpoint inhibitor, the patient did not respond and/or exhibited disease progression, in response to the FGFR inhibitor. According to particular embodiments, prior to the step of administering the immune checkpoint inhibitor, the patient did no longer respond to the FGFR inhibitor or the response to the FGFR inhibitor decreased.

According to particular embodiments, prior to the step of administering the FGFR inhibitor, the patient was treated with a first immune checkpoint inhibitor (prior to treatment with the FGFR inhibitor) and exhibited disease progression in response to said first immune checkpoint inhibitor (thus, in accordance with this embodiment, the patient did not previously respond to an immune checkpoint inhibitor, but is re-treated or “re-challenged” with the checkpoint inhibitor following exposure to the FGFR inhibitor).

According to particular embodiments, the immune checkpoint inhibitor is an antibody that blocks the interaction between PD-1 and PD-L1, such as pembrolizumab, atezolizumab, nivolumab, cetrelimab, or the like. It is alternatively an antibody that blocks the interaction between CTLA-4 and CD80 or CD86 on the surface of antigen-presenting cells. Non-limiting examples of immune checkpoint inhibitors that may be suitable in accordance with the present invention include atezolizumab, pembrolizumab, nivolumab, durvalumab, avelumab, anti-CSF1R antibody, tremelimumab, ipilimumab and the like.

According to particular embodiments, the patient has been diagnosed with bladder cancer, such as locally advanced or metastatic urothelial cancer; or locally advanced or metastatic urothelial cancer harboring FGFR2 or FGFR3 genetic alterations; or locally advanced or metastatic urothelial cancer harboring FGFR2 or FGFR3 genetic alterations and who progressed during or following at least one line of prior platinum-containing chemotherapy including within 12 months of neoadjuvant or adjuvant platinum-containing chemotherapy.

According to particular embodiments, the FGFR variant is selected from the group consisting of FGFR2:AFF3; FGFR2:BICC1; FGFR2:CASP7; FGFR2:CCDC6; FGFR2:OFD1; FGFR3:BAIAP2L1; FGFR3:TACC3-Intron; FGFR3:TACC3v1; FGFR3:TACC3v3; and a combination thereof; in particular FGFR2:BICC1; FGFR2:CASP7; FGFR3:BAIAP2L1; FGFR3:TACC3v1; FGFR3:TACC3v3 and a combination thereof.

According to particular embodiments, the FGFR variant is a FGFR3 mutation, in particular a FGFR 3 mutation selected from the group consisting of FGFR3 R248C; FGFR3 S249C; FGFR3 G370C; FGFR3 Y373C; and a combination thereof.

According to particular embodiments, the FGFR inhibitor is erdafitinib and is administered in an amount of between about 8 mg and about 9 mg daily.

According to particular embodiments, the method is effective in achieving a complete or partial response in the patient, e.g., in reducing a tumor volume in the patient and/or stopping or reducing disease progression.

In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### BRIEF DESCRIPTION OF THE DRAWINGS

Embodiments of the invention may be further understood when read in conjunction with the appended figures.

FIG. 1 is a graph illustrating response rate with prior systemic therapies.

FIG. 2 is a Kaplan-Meier plot of PFS following subsequent therapy to erdafitinib.

FIG. 3 is a Kaplan-Meier plot of OS following subsequent therapy to erdafitinib.

FIGS. 4a, 4b and 4c illustrate proportions of T cells compared to baseline in a Phase 1b-2 study to evaluate safety, efficacy, pharmacokinetics, and pharmacodynamics of erdafitinib plus cetrelimab.

FIGS. 5a, 5b and 5c illustrate proportions of T cells compared to baseline in a Phase 1b-2 study to evaluate safety, efficacy, pharmacokinetics, and pharmacodynamics of erdafitinib plus cetrelimab

FIGS. 6a and 6b illustrate proportions of T cells compared to baseline in a Phase 1b-2 study to evaluate safety, efficacy, pharmacokinetics, and pharmacodynamics of erdafitinib plus cetrelimab

### DETAILED DESCRIPTION

Immune Checkpoint Inhibitors

Immune checkpoint inhibitors (CPI) may revive pre-existing immune responses that are suppressed in certain solid tumors. The conventional paradigms in combining chemotherapy with CPIs has become the standard of care in first-line non-small cell lung cancer (NSCLC). This may indicate that conventional cytotoxic agents may perturb or prime the tumor microenvironment in such a way to modulate T cell-mediated tumoricidal activity. Patients with solid tumors are more likely to respond to a CPI in NSCLC with high levels of tumor mutational burden (TMB) and in metastatic microsatellite instability-high (MSI-H) or mismatch repair deficient solid tumors such as colon or ovarian cancer. Some targeted agents may affect the expression of checkpoint inhibitory molecules such as PD-L1 on tumor cells or sensitize the tumor to immune-mediated killing via alternate mechanisms. The BRAF inhibitor vemurafenib has been shown to increase expression of tumor antigens gp100 and MART1, increase tumor T cell infiltration, and decrease tumor secretion of immunosuppressive cytokines, and PD-L1 expression (See Hughes et al., Targeted Therapy and Checkpoint Immunotherapy Combinations for the Treatment of Cancer, Trends Immunol. 2016 Jul;37(7):462-476., and Vanneman et al., Combining immunotherapy and targeted therapies in cancer treatment, Nat Rev Cancer. 2012 Mar 22;12(4):237-51, which are incorporated by reference herein).

It has also become increasingly apparent that prior immunomodulation can prime solid tumors to respond to conventional cytotoxic therapies such as chemotherapy and targeted agents. The response to conventional therapies (cytotoxics and targeted) following progression after CPI would suggest a unique synergism within the tumor microenvironment. Improved response rates to systemic chemotherapy post-CPI has been described in several case series in NSCLC with patients showing unexpectedly high response rates (RR) with subsequent chemotherapy post-CPI (See Schvartsman et al. Lung Cancer 2017, Park et al. J Thorac Oncol. 2018, Grigg et al. J

Clin Oncol. 2017).

Urothelial Carcinoma and Immunotherapy

Urothelial carcinoma exhibits the third-highest mutation rate of all studied cancer types, behind NSCLC and melanoma (See Alexandrov et al., Signatures of mutational processes in human cancer, Nature. 2013 Aug 22;500(7463):415-21). High tumor mutation burden is predicted to correlate with response to immunotherapies, due to the generation of neoantigens which may be recognized by the immune system. Recently, checkpoint inhibitors including atezolizumab, pembrolizumab, nivolumab, durvalumab, and avelumab have been approved for treatment of advanced urothelial carcinoma, with observed response rates of—13-30%. Despite these improvements, however, most patients fail to benefit from checkpoint inhibition. The response to checkpoint inhibitors is largely dependent on an existing anti-tumor T cell response, including sufficient T cell infiltration in the tumor microenvironment (See Harlin et al., Chemokine expression in melanoma metastases associated with CD8+ T-cell recruitment, Cancer Res. 2009 Apr 1;69(7):3077-85). However, not all urothelial cancers exhibit high T cell infiltration. A report classified the microenvironment of urothelial carcinoma tumors as T-cell-inflamed versus non-T-cell-inflamed. FGFR mutations were significantly enriched in the non-T-cell-inflamed group, with no FGFR pathway alterations identified in T-cell-inflamed samples (See Sweis et al., Molecular Drivers of the Non-T-Cell-Inflamed Tumor Microenvironment in Urothelial Bladder Cancer, Cancer Immunol Res. 2016 July;4(7):563-8). Differential responses to immunotherapies have been observed in urothelial carcinoma based on bladder cancer molecular subtype, and the underlying immune landscape of these subtypes. Urothelial cancer, like breast cancer, can be classified via gene expression signature into luminal and basal subtypes (luminal 1, 2, or basal 3, 4). Luminal 1 tumors are reported to be enriched for FGFR3 mutations, and lacking in immune marker expression and immne cell infiltrate. The luminal 1 subtype showed the lowest response rate to the anti-PD-(L)1 inhibitors atezolizumab and nivolumab compared with other bladder cancer subtypes. Analyses of atezolizumab Phase 2 data showed PD-L1 expression on tumor infiltrating immune cells was more pronounced in the basal subtype compared with the luminal subtype, with response to atezolizumab lowest in the luminal 1 group.

Immune Priming with Erdafitinib

Erdafitinib is an FGFR-kinase inhibitor approved by the U.S. Food and Drug Administration for the treatment of adults with locally advanced or metastatic urothelial carcinoma (mUC) harboring susceptible FGFR3 or FGFR2 genetic alterations and who progressed during or following at least one line of prior platinum-containing chemotherapy including within 12 months of neoadjuvant or adjuvant platinum-containing chemotherapy. The present invention provides improved treatment regimens involving FGFR inhibitors, such as erdafitinib, in this distinct and molecularly-defined population of FGFR-positive patients with mUC.

The chemical name of erdafitinib is N-(3,5-dimethoxyphenyl)-N′-(1-methylethyl)-N-[3-(1-methyl-1H-pyrazol-4-yl)quinoxalin-6-yl]ethane-1,2-diamine and the chemical structure is as follows:

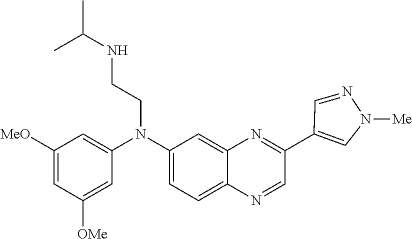 [SMILES: COc1cc(OC)cc(N(CCNC(C)C)c2ccc3ncc(-c4cnn(C)c4)nc3c2)c1]

The clinical evidence described herein indicate that erdafitinib may not only prime the immune system in FGFR-driver alterations such as mutations and fusions in solid tumors, but also enhance subsequent anti-tumor responses when sequentially exposed to an immune checkpoint inhibition (CPI). As described in the example below, it was unexpectedly observed in a clinical setting that treatment of a patient with erdafitinib improved the patient's subsequent response to an immune checkpoint inhibitor. In that Phase 2 study, it was observed that urothelial carcinoma patients with FGFR alterations are less likely to respond to checkpoint inhibitors: only 1/22 patients responded to prior immunotherapy and that prior response was in combination with another experimental therapy. However, response rates for subsequent immunotherapy after erdafitinib treatment had higher objective response rate (ORR) and disease control rate (DCR) versus patients who received chemotherapy after erdafitinib (see Table 5). Thus, it is believed that erdafitinib enhanced patient sensitivity to immunotherapy with a checkpoint inhibitor. Stated another way, the effect of immunotherapy after treatment with erdafitinib was better than the effect of immunotherapy without prior administration of erdafitinib.

According to an embodiment, a method of treating cancer in a patient comprises administering an immune checkpoint inhibitor to the patient, wherein the patient has an FGFR variant, and has been pre-treated with an FGFR inhibitor, such as erdafitinib. According to an embodiment, there is provided an immune checkpoint inhibitor for use in the treatment of cancer in a patient, wherein the patient has an FGFR variant, and has been pre-treated with an FGFR inhibitor, such as erdafitinib. According to an embodiment, there is provided use of an immune checkpoint inhibitor for the manufacture of a medicament for the treatment of cancer in a patient, wherein the patient has an FGFR variant, and has been pre-treated with an FGFR inhibitor, such as erdafitinib.

A patient is determined to have an FGFR variant (i.e., FGFR genetic alteration) if a biological sample of the patient has tested positive for the presence of one or more FGFR variants, in particular one or more FGFR mutations or fusions, more in particular one or more FGFR2 or FGFR3 mutations or fusions or one or more FGFR3 mutations or FGFR2 or FGFR3 fusions.

As used herein, “biological sample” refers to any sample from a patient in which cancerous cells can be obtained, e.g., from tumor tissue biopsy or a liquid biopsy from circulating tumor DNA (CT-DNA) or circulating tumor cells (CTC) where DNA and/or RNA can be isolated. Suitable biological samples can include, but are not limited to, blood, lymph fluid, bone marrow, sputum, a solid tumor sample, or any combination thereof. In some embodiments, the biological sample can be formalin-fixed paraffin-embedded tissue (FFPET).

As used herein, “FGFR variant” refers to an alteration in the wild type FGFR gene, including, but not limited to, FGFR fusion genes, FGFR mutations, FGFR amplifications, or any combination thereof “FGFR fusion” or “FGFR fusion gene” refers to a gene encoding a portion of FGFR (e.g., FGRF2 or FGFR3) and one of the herein disclosed fusion partners created by a translocation between the two genes. An “FGFR-altered cancer” or “FGFR-genetically altered cancer” is a cancer in which the patient has been diagnosed with a solid tumor cancer and one or more FGFR variants are present in a biological sample from the patient.

As used herein, “patient” is intended to mean any animal, in particular, mammals. Thus, the methods are applicable to human and nonhuman animals, although most preferably with humans. “Patient” and “subject” may be used interchangeably herein.

As used herein, “has been treated with a FGFR inhibitor” or “has been pre-treated with an FGFR inhibitor” is intended to mean that the patient received treatment with a FGFR inhibitor prior to the treatment with an immune checkpoint inhibitor. According to an embodiment, the patient continues to receive treatment with the FGFR inhibitor while on treatment with the immune checkpoint inhibitor. According to another embodiment, the patients discontinues treatment with the FGFR inhibitor while on treatment with the immune checkpoint inhibitor. In certain embodiments described herein, the patients received one or more cancer treatments prior to the treatment with the FGFR inhibitor, including for example chemotherapy or an immune checkpoint inhibitor.

Exemplary FGFR inhibitors are described in U.S. Publ. No. 2013/0072457 A1 (incorporated herein by reference) and include N-(3,5-dimethoxy-phenyl)-N′-(1-methylethyl)-N-[3-(1-methyl-1H-pyrazol-4-yl-)quinoxalin-6-yl] ethane-1,2-diamine (referred to herein as erdafitinib), including any N-oxide thereof, any pharmaceutically acceptable salt thereof, or any solvate thereof. Thus, in some embodiments, the FGFR inhibitor can be erdafitinib or a pharmaceutically acceptable salt thereof. In some aspects, the pharmaceutically acceptable salt is a HCl salt. In some aspects, the FGFR inhibitor is erdafitinib free base.

The disclosed methods or uses are suitable for treating cancer in a patient if one or more FGFR variants are present in a biological sample from the patient. In some embodiments, the FGFR variant can be one or more FGFR fusion genes. In some embodiments, the FGFR variant can be one or more FGFR mutations. In some embodiments, the FGFR variant can be one or more FGFR amplifications. In some embodiments, a combination of the one or more FGFR variants can be present in the biological sample from the patient. For example, in some embodiments, the FGFR variants can be one or more FGFR fusion genes and one or more FGFR mutations. In some embodiments, the FGFR variants can be one or more FGFR fusion genes and one or more FGFR amplifications. In some embodiments, the FGFR variants can be one or more FGFR mutations and one or more FGFR amplifications. In yet other embodiments, the FGFR variants can be one or more FGFR fusion genes, mutations, and amplifications.

Examplary FGFR variants are described in, for example, U.S. Publication No. 2019/0078166, which is incorporated by reference herein. Exemplary FGFR fusion genes include, but are not limited to: FGFR2:AFF3; FGFR2:BICC1; FGFR2:CASP7; FGFR2:CCDC6; FGFR2:OFD1; FGFR3:BAIAP2L1; FGFR3:TACC3-Intron; FGFR3:TACC3v1; FGFR3:TACC3v3; or a combination thereof. Exemplary FGFR mutations include, but are not limited to, FGFR3 R248C; FGFR3 S249C; FGFR3 G370C; FGFR3 Y373C; or a combination thereof.

The methods or uses described herein may further comprise evaluating the presence of one or more FGFR variants in the biological sample before the administering step(s), in particular the FGFR inhibitor administering step. Suitable methods for evaluating a biological sample for the presence of one or more FGFR variants are disclosed, for example, in U.S. Publication No. 2019/0078166 and U.S. Publication No. 2016/0090633, which are incorporated by reference herein. For example, and without intent to be limiting, evaluating a biological sample for the presence of one or more FGFR variants can comprise any combination of the following steps: isolating RNA from the biological sample; synthesizing cDNA from the RNA; and amplifying the cDNA (preamplified or non-preamplified). According to particular embodiments, evaluating a biological sample for the presence of one or more FGFR variants comprises next generation sequencing (NGS) or real-time polymerase chain reaction (RT-PCR). In some aspects, the cDNA can be pre-amplified. In some aspects, the evaluating step can comprise isolating RNA from the sample, synthesizing cDNA from the isolated RNA, and pre-amplifying the cDNA.

Enbodiments of the present invention relate to the use of an FGFR inhibitor (e.g., erdafitinib) to prime, sensitize, and enhance a cancer patient's subsequent response to an immune checkpoint inhibitor. Such patients treated with erdafitinib have a FGFR-genetic alteration. Enhanced responses may be seen by the physician, for example, if the patient previously had disease progression on an immune checkpoint inhibitor and was re-challenged with an immune checkpoint inhibitor after treatment with the FGFR inhibitor and responded; or if that cancer typically has low response rates to immune checkpoint inhibitors from the clinical literature.

Additional enbodiments of the present invention relate to the use of an immune checkpoint inhibitor for the treatment of a cancer patient following disease progression after treatment with an FGFR inhibitor (e.g., erdafitinib). According to an aspect, there is provided an immune checkpoint inhibitor for the treatment of cancer in a patient following disease progression after treatment with an FGFR inhibitor (e.g., erdafitinib). Disease progression may be determined, for example, via radiographic evidence of tumor enlargement by RECIST criteria or radiologist impression and/or symptomatic evidence if the patient's clinical condition is declining rapidly from the cancer despite treatment.

According to an embodiment, a method of treating cancer in a patient comprises administering a therapeutically effective amount of an immune checkpoint inhibitor to the patient, wherein the patient has an FGFR variant, and has been treated with an FGFR inhibitor. Stated another way, the method may comprise administering an FGFR inhibitor to the patient for a first period of time, and after said period of time, administering an immune checkpoint inhibitor to the patient for a subsequent period of time.

According to particular embodiments, the FGFR inhibitor is erdafitinib or a pharmaceutically acceptable salt thereof, in particular erdafitinib free base.

According to particular embodiments, prior to the step of administering the immune checkpoint inhibitor, the patient exhibited disease progression in response to the FGFR inhibitor.

According to particular embodiments, prior to the step of administering the FGFR inhibitor, the patient was treated with a first immune checkpoint inhibitor and exhibited disease progression in response to said first immune checkpoint inhibitor. Thus, according to these embodiments, the patient did not respond to the “first immune checkpoint inhibitor,” but following treatment (e.g., sensitization) with the FGFR inhibitor, the patient responded to the subsequent immune checkpoint inhibitor (which may be the same or different compound as the “first immune checkpoint inhibitor”).

According to particular embodiments, the immune checkpoint inhibitor is an antibody that blocks the interaction between PD-1 and PD-L1, such as pembrolizumab or atezolizumab or nivolumab. According to alternative embodiments, the immune checkpoint inhibitor is an antibody that blocks the interaction between CTLA-4 and CD80 or CD86. According to particular embodiments, the immune checkpoint inhibitor is cetrelimab.

According to particular embodiments, the patient has been diagnosed with an FGFR-genetically altered solid tumor. For example, the tumor may be located in the breast, lung or bladder.

According to particular embodiments, the patient has been diagnosed with bladder cancer, such as locally advanced or metastatic urothelial cancer; or locally advanced or metastatic urothelial cancer harboring FGFR2 or FGFR3 genetic alterations; or locally advanced or metastatic urothelial cancer harboring FGFR2 or FGFR3 genetic alterations and who progressed during or following at least one line of prior platinum-containing chemotherapy including within 12 months of neoadjuvant or adjuvant platinum-containing chemotherapy.

According to particular embodiments, the FGFR variant is selected from the group consisting of FGFR2:AFF3; FGFR2:BICC1; FGFR2:CASP7; FGFR2:CCDC6; FGFR2:OFD1; FGFR3:BAIAP2L1; FGFR3: TACC3-Intron; FGFR3:TACC3v1; FGFR3:TACC3v3; and a combination thereof. In particular, the FGFR variant is selected from the group consisting of FGFR2:BICC1; FGFR2:CASP7; FGFR3:BAIAP2L1; FGFR3:TACC3v1; FGFR3:TACC3v3; and a combination thereof.

According to particular embodiments, the FGFR variant is selected from the group consisting of FGFR3 R248C; FGFR3 S249C; FGFR3 G370C; FGFR3 Y373C; and a combination thereof.

According to particular embodiments, the immune checkpoint inhibitor is an antibody that blocks the interaction between PD-1 and PD-L1.

According to particular embodiments, the immune checkpoint inhibitor is an antibody that blocks the interaction between CTLA-4 CD80 or CD86 on the surface of antigen-presenting cells, such as ipilitumab.

According to particular embodiments, the immune checkpoint inhibitor is pembrolizumab, atezolizumab, cetrelimab, nivolumab, durvalumab, avelumab, ipilimumab, anti-CSF1R antibody, tremelimumab.

According to particular embodiments, the FGFR inhibitor is erdafitinib and is administered in an amount of between about 8 mg and about 9 mg daily.

According to particular embodiments, the method is effective in achieving a complete or partial response in the patient, e.g., in reducing a tumor volume in the patient and/or stopping or reducing disease progression.

According to particular embodiments, the method or use comprises administering the FGFR inhibitor systemically (e.g., via oral administration of a tablet).

According to particular embodiments, the present invention provides a method of treating a patient diagnosed with cancer by administering an FGFR inhibitor (e.g., erdafitinib) in an amount effective to sensitize the patient to an immune checkpoint inhibitor, the method comprising:

administering an FGFR inhibitor to the patient during a second period of time, wherein an immune checkpoint inhibitor is not administered to the patient during said second period of time; and

after said second period of time, administering an immune checkpoint inhibitor to the patient during a third period of time, wherein an FGFR inhibitor is not administered to the patient during said third period of time, wherein the patient:

(a) has been diagnosed with an FGFR-genetically altered cancer,

(b) was administered a first immune checkpoint inhibitor during a first period of time prior to the second period of time, wherein an FGFR inhibitor was not administered to the patient during said first period of time, and

(c) did not respond to the first immune checkpoint inhibitor during said first period of time (e.g., exhibited disease progression).

According to particular embodiments, the patient responded to the FGFR inhibitor during said second period of time (e.g., disease progression was stopped or reduced).

According to certain embodiments, the patient's response to the immune checkpoint inhibitor administered during the third period of time (following administration of the FGFR inhibitor) is greater than the patient's response to an immune checkpoint inhibitor in the absence of pre-treatment with an FGFR inhibitor (e.g., during or following the first period of time). It is believed that FGFR-genetically altered tumors may be “immunologically-cold” or refractory to I/O therapy; however, the tumors may be rendered “hot”, i.e., sensitized to immune checkpont inhibitors, following exposure to an FGFR inhibitor such as erdafitinib during said second period of time, so that the patient responds to the immune checkpoint inhibitor administered during the third period of time.

According to particular embodiments, the present invention provides a method of treating a patient diagnosed with cancer by administering an FGFR inhibitor (e.g., erdafitinib) in an amount effective to sensitize the patient to an immune checkpoint inhibitor, the method comprising:

administering an FGFR inhibitor to the patient during a first period of time, wherein an immune checkpoint inhibitor is not administered to the patient during said first period of time; and after said first period of time, administering an immune checkpoint inhibitor to the patient during a second period of time, wherein an FGFR inhibitor is not administered to the patient during said second period of time, wherein the patient has been diagnosed with an FGFR-genetically altered cancer. According to particular embodiments, the present invention provides an FGFR inhibitor (e.g., erdafitinib) for use in the treatment of cancer in a patient diagnosed with cancer by administering an FGFR inhibitor (e.g., erdafitinib) in an amount effective to sensitize the patient to an immune checkpoint inhibitor, the method comprising:

administering an FGFR inhibitor to the patient during a first period of time, wherein an immune checkpoint inhibitor is not administered to the patient during said first period of time; and

after said first period of time, administering an immune checkpoint inhibitor to the patient during a second period of time, wherein an FGFR inhibitor is not administered to the patient during said second period of time, wherein the patient has been diagnosed with an FGFR-genetically altered cancer.

Additional embodiments of the present invention are provided below:

(1) Use of an immune checkpoint inhibitor (e.g., an antibody that blocks the interaction between PD-1 and PD-L1, such as pembrolizumab or atezolizumab) for the manufacture of a medicament for the treatment of a cancer patient that progressed after treatment with a FGFR inhibitor (e.g., erdafitinib).

(2) An immune checkpoint inhibitor (e.g., an antibody that blocks the interaction between PD-1 and PD-L1, such as pembrolizumab or atezolizumab) for use in the treatment of a cancer patient that progressed after treatment with a FGFR inhibitor (e.g., erdafitinib).

(3) Use of an immune checkpoint inhibitor (e.g., an antibody that blocks the interaction between PD-1 and PD-L1, such as pembrolizumab or atezolizumab or nivolumab; or an antibody that blocks the interaction between CTLA-4 and CD80 or CD86) for the manufacture of a medicament for the treatment of a cancer patient wherein the patient is a patient that progressed after treatment with a FGFR inhibitor (e.g., erdafitinib) and wherein the patient received the FGFR inhibitor after a biological sample of the cancer patient tested positive for the presence of one or more FGFR variants, in particular one or more FGFR mutations and/or fusions.

(4) An immune checkpoint inhibitor for use in the treatment of a cancer patient wherein the patient is a patient that progressed after treatment with a FGFR inhibitor and wherein the patient received the FGFR inhibitor after a biological sample of the cancer patient tested positive for the presence of one or more FGFR variants, in particular one or more FGFR mutations and/or fusions

(5) Use of a FGFR inhibitor to sensitize a cancer patient to an immune checkpoint inhibitor.

(6) A FGFR inhibitor for use in sensitizing a cancer patient to an immune checkpoint inhibitor.

(7) Use of a FGFR inhibitor to re-sensitize a cancer patient to an immune checkpoint inhibitor.

(8) A FGFR inhibitor for use in re-sensitizing a cancer patient to an immune checkpoint inhibitor.

(9) A FGFR inhibitor for use in a treatment sequence wherein a cancer patient is re-challenged to an immune checkpoint inhibitor wherein the cancer patient had disease progression on a previous immune checkpoint inhibitor.

(10) A FGFR inhibitor for use in a treatment sequence wherein a cancer patient is re-challenged to an immune checkpoint inhibitor after the patient had disease progression when on treatment with the FGFR inhibitor and wherein the cancer patient had previous disease progression on a previous immune checkpoint inhibitor.

(11) Use of a FGFR inhibitor for the preparation of a medicament for the treatment of a cancer patient wherein the FGFR inhibitor is used in a treatment sequence wherein the cancer patient is re-challenged to an immune checkpoint inhibitor after treatment with the FGFR inhibitor and wherein the cancer patient had disease progression on a previous immune checkpoint inhibitor.

(12) Use of a FGFR inhibitor for the preparation of a medicament for the treatment of a cancer patient wherein the FGFR inhibitor is used in a treatment sequence wherein the cancer patient is re-challenged to an immune checkpoint inhibitor after the patient had disease progression when on treatment with the FGFR inhibitor and wherein the cancer patient had previous disease progression on a previous immune checkpoint inhibitor.

The term “therapeutically effective amount” refers to an amount (e.g., of an active compound or pharmaceutical agent, such as a FGFR inhibitor, e.g. erdafitinib, or an immune checkpoint inhibitor), which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease. Stated another way, the term therapeutically effective amount may refer to an amount that, when administered to a particular subject, achieves a therapeutic effect by inhibiting, alleviating or curing a disease, condition, syndrome or disorder in the subject or by prophylactically inhibiting, preventing or delaying the onset of a disease, condition, syndrome or disorder, or symptom(s) thereof. A therapeutically effective amount may be an amount which relieves to some extent one or more symptoms of a disease, condition, syndrome or disorder in a subject; and/or returns to normal either partially or completely one or more physiological or biochemical parameters associated with or causative of the disease, condition, syndrome or disorder; and/or reduces the likelihood of the onset of the disease, condition, syndrome or disorder, or symptom(s) thereof.

The term “FGFR inhibitor” as used herein refers to a chemical compound that inhibits enzymatic activity of one or more fibroblast growth factor receptors (FGFRs), such as FGFR1, FGFR2, FGFR3, FGFR4.

According to particular embodiments, efficacy of the methods described herein is measured by determining a patient time to disease progression or a patient response rate. In some embodiments, efficacy is measured by determining the patient's time to disease progression, e.g., a reduction in disease progression over time in response to treatment according to a method of the present disclosure. The disease progression may be measured by proliferation of the cancer cells (locally or systemically), and/or reoccurrence of side effects of the disease, and/or occurrence of new side effects of the disease. In other embodiments, the efficacy is measured by determining a patient response rate. The “response rate” as used herein is the ratio of the number patients who respond to treatment (by a demonstration of efficacy) to the number of patients who have been treated. According to particular embodiments, the efficacy of a treatment method of the present disclosure is measured by one or more of decrease in proliferation of the cancer cells (locally or systemically), the absence of cancer cells (locally or systemically), decrease of side effects of the disease, or elimination of side effects of the disease. According to particular embodiments, a method or use of the present invention is effective in reducing a tumor volume in the patient following treatment. Evaluation of a patient's tumor response may be made according to known criteria referred to as Response Evaluation Criteria in Solid Tumors (RECIST) 1.1.

The methods or uses described herein permit administration of the FGFR inhibitor via any acceptable route. In some embodiments, the FGFR inihibitor is administered orally, parenterally (i.e., in the form of a liquid), rectally (i.e., in the form of a suppository), topically (i.e., in the form of a transdermal patch, ointment, or cream), or intranasally. Examples of parenteral administration include intravenous (IV), intramuscular (IM), and subcutaneous (SC) injection. Preferably, the FGFR inhibitor is administered orally, in particular once daily.

According to particular embodiments, the immune checkpoint inhibitor is administered intraveneously.

While it is possible for the active ingredient to be administered alone, i.e., neat, it may also be present in pharmaceutical composition. Accordingly, the present disclosure further provides a pharmaceutical composition and, as active ingredient, the FGFR inhibitor described herein. As such the FGFR inhibitor may be formulated into various pharmaceutical forms for any conventional routes of administration.

When the FGFR inhibitor is formulated in a pharmaceutical composition, the composition also comprises one or more pharmaceutically acceptable carrier(s), diluent(s), and/or excipient(s). The particular carrier, diluent, and/or excipient will depend on the route of administration and may be determined by those skilled in the art. The carrier, diluent, and/or excipient must be “acceptable” in the sense of being compatible with the other ingredients of the composition and not deleterious to the recipients thereof. Examples of excipients include diluents, lubricants, binders, and disintegrating agents. suspending agents, penetration enhancing agent and/or a suitable wetting agent. The excipient may be in the form of a liquid such as water, a glycol, an oil, or an alcohol or a solid such as a starch, sugar, or kaolin.

According to an embodiment, erdafitinib is formulated as a tablet for oral administration. The table may comprise excipients selected from croscarmellose sodium, magnesium stearate, mannitol, meglumine, microcrystalline cellulose, and the like. According to an embodiment, erdafitinib is formulated as a tablet comprising 3 mg base equivalent of erdafitinib. According to an embodiment, erdafitinib is formulated as a tablet comprising 4 mg base equivalent of erdafitinib. According to an embodiment, erdafitinib is formulated as a tablet comprising 5 mg base equivalent of erdafitinib. According to an embodiment, erdafitinib is administered in a dose of 8 mg daily, in particular once daily, in particular as 2 times a tablet comprising 4 mg base equivalent of erdafitinib. According to an embodiment, erdafitinib is administered in a dose of 9 mg daily, in particular once daily, in particular as 3 times a tablet comprising 3 mg base equivalent of erdafitinib.

Pharmaceutical compositions designed for oral administration may be in the form of solid or liquid. In some embodiments, the oral formulation is a liquid preparation such as a suspension, syrup, elixir, emulsion, or solution. In other embodiments, the oral formulation is a solid preparation such as a tablet (including scored or coated tablets), capsule, caplet (including scored or coated caplets), pill, powder, or wafer.

### Examples

### Example 1. Clinical Study

In this analysis, clinical responses to prior and subsequent therapies in FGFR-positive patients with mUC from a pivotal phase 2 study of erdafitinib were evaluated.

Study Overview:

This is a retrospective analysis of data collected from patients randomized to regimen 3 (8 mg once daily erdafitinib) of the phase 2, multicenter, open-label study (BLC2001; NCT02365597) of erdafitinib. The phase 2 study is described, for example, in Loriot Y, et al. N Engl J Med. 2019;25;381(4):338-348, which is incorporated by reference herein.

Patients had prespecified FGFR2/3 mutations/fusions, locally advanced and unresectable or metastatic urothelial carcinoma, and progression during or after ≥1 line prior chemotherapy or within 12 months of adjuvant/neoadjuvant chemotherapy, or were cisplatin ineligible and chemotherapy naïve.

Prior systemic therapies received for metastatic or surgically unresectable UC and subsequent lines of treatment to erdafitinib were reported by the investigator.

The study endpoints included the following:

1. Duration of prior treatment: time interval from start of first dose of current therapy line to first dose of next therapy line for prior treatments.

2. Time to progression (TTP): time interval from initiation of a prior therapy to disease progression on that same therapy.

3. Response to prior therapies: responses were evaluated using the investigator reported best response.
a) Objective response rate (ORR): percentage of patients with complete and partial response to treatment
b) Disease control rate (DCR): percentage of patients with complete, partial and stable disease responses

6. Progression-free survival (PFS): time interval from initiation of first study dose/subsequent therapy until disease progression or death due to any cause, whichever occurred first

7. Overall survival (OS): time interval from initiation of first study dose/subsequent therapy until death due to any cause

8. Response to subsequent therapies was assessed using investigator reported best response to calculate ORR and DCR.

Responses were assessed by investigators and response rates were summarized using frequency and percentages. The Kaplan-Meier method was used to estimate survival outcomes (TTP, PFS and OS) and median values along with 95% CI were provided.

Of 210 eligible patients, 99 were enrolled in the 8 mg (uptitration to 9 mg) once-daily erdafitinib group.

TABLE 1
Demographics and baseline characteristics

| | Erdafitinib |
|---|---|
| | (8 mg once-daily*) |
| Characteristic | n = 99 |
| Age, median (range), years | 68 (36-87) |
| Sex, n (%) | |
| ECOG performance status, n (%) | 76 (77) |
| 0 | 50 (51) |
| 1 | 42 (42) |
| 2 | 7 (7) |
| Pre-treatment status, n (%) | |
| Chemotherapy relapsed/refractory | 87 (87.9) |
| Chemotherapy-naïve | 12 (12.1) |
| No. of lines of prior treatment | |
| 0 | 11 (11) |
| 1 | 45 (46) |
| 2 | 29 (29) |
| 3 | 10 (10) |
| >3 | 4 (4) |
| Time from initial diagnosis to 1st dose of | 24.74 (1.6-288.9) |
| erdafitinib (range), months | |
| Time from progression/relapse on the last | 1.64 (0.2-33.4) |
| line of treatment to 1st dose of erdafitinib, | |
| median (range), months | |

*Patients whose serum phosphate was <5.5 mg/dL on day 14 of cycle 1 and had no erdafitinib-related toxicity were uptitrated to 9 mg daily
ECOG, Eastern Cooperative Oncology Group

In total, 88/99 (88.9%) patients received prior systemic therapies (Table 2). In total, 34/99 (34.3%) patients received subsequent systemic therapy following treatment with erdafitinib; 19/99 (19.2%) received subsequent chemotherapy and 15/99 (15.2%) received subsequent immunotherapy.

TABLE 2
Prior and subsequent treatments

| | Erdafitinib |
|---|---|
| | (8 mg once-daily^{a}) |
| | n = 99 |
| Prior treatments, n (%) | 88 (88.9) |
| Prior chemotherapy | |
| 1^{st} line containing chemotherapy | 84 (84.8) |
| 1^{st} line containing G-C | 44 (44.4) |
| 1^{st} line containing G-Cb | 30 (30.3) |
| 1^{st} line containing MVAC | 8 (8.1) |
| Other platinum combinations | 2 (2.0) |
| 2^{nd} line containing chemotherapy | |
| 2^{nd} line containing docetaxel | 16 (16.2) |
| 3rd line containing chemotherapy | |
| 3rd line containing docetaxel | 7 (7.1) |
| Any prior immunotherapy^{b, c} | |
| Containing pembrolizumab or nivolumab | 4 (4.0) |
| Containing atezolizumab, durvalumab or | 18 (18.2) |
| avelumab | |
| Subsequent therapy, n (%) | 34 (34.3) |
| Number of therapy lines | |
| 1 | 25 (25.3) |
| 2 | 9 (9.1) |
| Subsequent chemotherapy | 19 (19.2) |
| Subsequent immunotherapy^{d} | 15 (15.2) |

^{a}Patients whose serum phosphate was <5.5 mg/dL on day 14 of cycle 1 and had no erdafitinib-related toxicity were uptitrated to 9 mg daily.
^{b}Prior immunotherapy includes atezolizumab, pembrolizumab, nivolumab, durvalumab, avelumab, anti-CSF1R antibody, tremelimumab.
^{c}2 patients received other types of immunotherapy in combination with anti-PD(L)-1 therapy: n = 1, durvalumab + tremelimuman, n = 1, atezolizumab + anti-CSF1R.
^{d}Includes atezolizumab (n = 2), pembrolizumab (n = 3), nivolumab (n = 5), durvalumab (n = 3), ipilimumab (n = 2).
Cb = carboplatin
C = cisplatin
MVAC = methotrexate/vinblastine/doxorubicin or epirubicin/cisplatin
G = gemcitabine

The median duration of erdafitinib treatment in the BLC2001 study was 5.3 months. See Loriot Y, et al. N Engl J Med. 2019;25;381(4):338-348, which is incorporated by reference herein.

TABLE 3
Median duration of prior systemic therapies

| | Erdafitinib |
|---|---|
| | (8 mg daily +/− uptitration^{a}) |
| | Median duration (95% CI), |
| | months |
| 1^{st} line chemotherapy, n = 84 | 9.35 (7.89; 10.41) |
| 2^{nd} line chemotherapy, n = 31 | 9.23 (5.39; 11.50) |
| 3^{rd} line chemotherapy, n = 10 | 6.26 (1.48; 8.38) |
| Any prior immunotherapy, n = 22 | 8.43 (4.63; 14.46) |
| 1^{st} line therapy in patients | |
| stratified by FGFR status | |
| FGFR3 mutations, n = 65 | 9.03 (7.13; 10.41) |
| FGFR 2/3 fusions, n = 23 | 9.59 (5.62; 11.30) |

^{a}patients whose serum phosphate was <5.5 mg/dL on day 14 of cycle 1 and had no erdafitinib-related toxicity were uptitrated to 9 mg daily.
C, cisplatin;
FGFR, fibroblast growth factor receptor

Regarding the time to progression (TTP) on prior therapies: The median (95% CI) TTP for prior first-line therapy (7.34 [5.91; 8.80] months) was longer than the median TTP for second-(7.13 [3.78; 9.36] months) or third-line therapy (5.70 [2.33; 8.64] months).

TABLE 4
Median TTP on prior therapies

| | Erdafitinib |
|---|---|
| | (8 mg once-daily*) |
| TTP, median (95% CI), months | n = 99 |
| 1^{st}-line chemotherapy (n = 78) | 7.34 (5.91; 8.80) |
| 1^{st} line chemotherapy containing G-C (n = 40) | 8.99 (6.37, 10.61) |
| 1^{st} line chemotherapy containing G-Cb (n = 28) | 6.59 (3.84, 7.39) |
| 1^{st} line chemotherapy containing MVAC (n = 8) | 8.34 (2.23, 12.81) |
| 2^{nd}-line chemotherapy (n = 29) | 7.13 (3.78; 9.36) |
| 2^{nd}-line D/V/P (n = 15) | 7.13 (3.06; 10.48) |
| 3^{rd}-line chemotherapy (n = 8) | 5.70 (2.33; 8.64) |
| 3^{rd}-line D/V/P (n = 5) | 5.55 (2.99; 9.46) |
| Any prior immunotherapy (n = 20) | 5.55 (2.30; 11.53) |

Note:
TTP was calculated for patients with an available date of progression prior to study entry.
C, cisplatin;
Cb, carboplatin;
D/V/P, docetaxel/vinflunine/paclitaxel;
G, gemcitabine;
MVAC, methotrexate/vinblastine/doxorubicin/cisplatin or methotrexate/vinblastine/epirubicin/cisplatin;
TTP, time to progression
*Patients whose serum phosphate was <5.5 mg/dL on day 14 of cycle 1 and had no erdafitinib-related toxicity were uptitrated to 9 mg daily.

Regarding response rates for prior and subsequent therapies: In the BLC2001 study, treatment with erdafitinib showed a confirmed ORR of 40% (95% CI: 30.7; 50.1), per investigator assessment. It is noted that only 1 of 22 patients (<5%) responded to prior I/O therapy before being treated with erdafitinib. Furthermore, the 1 patient that responded to prior I/O therapy was in combination with an experimental I/O agent. In short, these observations support the clinical observation that FGFR-genetically altered tumors (luminal 1) may be “immunologically-cold” or refractory to I/O therapy.

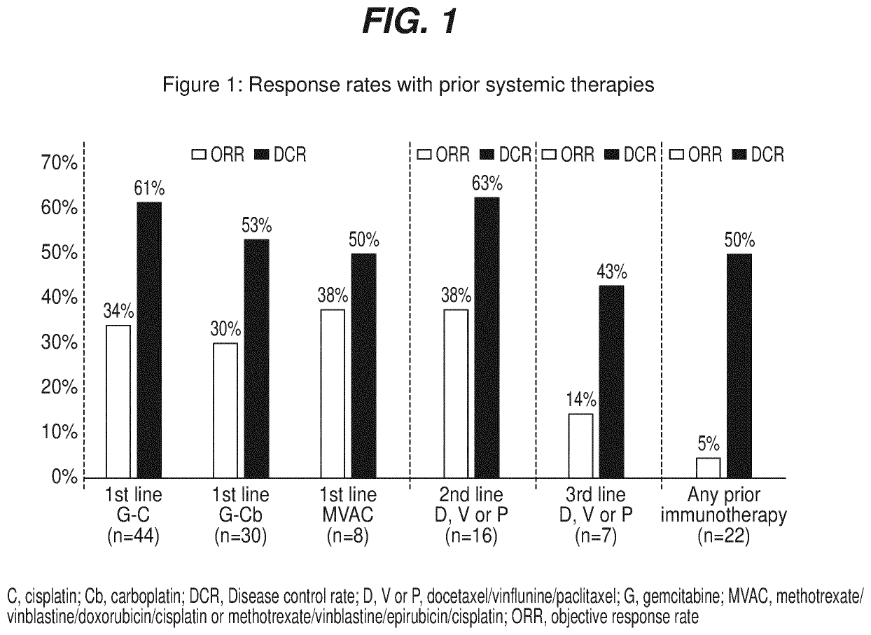

Higher ORR and DCR were observed with prior first-line and second-line chemotherapy as compared to third-line of chemotherapy. See FIG. 1.

The ORR and DCR with first-line prior systemic therapy for patients with FGFR3 mutations (n=65) vs. patients with FGFR2/3 fusions (n=23) were:

ORR: 20 (30.8%), 95% CI: 19.5; 42.0 vs. 8 (34.8%), 95% CI: 15.3 vs. 54.2

DCR: 40 (61.5%), 95% CI: 49.7; 73.4 vs. 11 (47.8%), 95% CI: 27.4, 68.2

Regarding response rates for subsequent therapies: patients on subsequent immunotherapy after erdafitinib treatment had higher ORR and DCR versus patients who received chemotherapy after erdafitinib. It is noted that the ORR prior to I/O therapy in the 22-patient cohort was less than 5%.

TABLE 5
Response rates with subsequent therapies

| | Erdafitinib | | | |
|---|---|---|---|---|
| | (8 mg daily +/− uptitration^{a}) | | | |
| | n = 99 | | | |
| | ORR, n (%) | | DCR, n (%) | |
| | [95% CI] | | [95% CI] | |
| Any 1^{st} subsequent therapy^{b} | 1 (2.9) | [0; 8.6] | 3 (8.8) | [0; 18.4] |
| (n = 34) | | | | |
| 1^{st} subsequent chemotherapy | 0 | [NE; NE] | 0 | [NE; NE] |
| (n = 16) | | | | |
| 1^{st} subsequent immunotherapy | 1 (6.7) | [0; 19.3] | 3 (20.0) | [0; 40.2] |
| (n = 15) | | | | |
| 2^{nd} subsequent therapy (n = 9) | 1 (11.1) | [0; 31.6] | 1 (11.1) | [0; 31.6] |
| 2^{nd} subsequent chemotherapy | 0 | [NE; NE] | 0 | [NE; NE] |
| (n = 7) | | | | |
| 2^{nd} subsequent immuno- | 1 (50.0) | [0; 100] | 1 (50.0) | [0; 100] |
| therapy (n = 2) | | | | |

^{a}patients whose serum phosphate was <5.5 mg/dL on day 14 of cycle 1 and had no erdafitinib-related toxicity were uptitrated to 9 mg daily;
^{b}n = 2 patients received 1st subsequent therapy containing radiotherapy.
CI, confidence interval; DCR, disease control rate; ME, not estimable; ORR, objective response rate.

Regarding progression-free survival (PFS) and overall survival (OS) following subsequent therapy:

In the BLC2001 study, the median PFS was 5.5 months (95% CI: 4.2; 6.0) and median OS was 13.8 months (95% CI: 9.8; not reached) with erdafitinib therapy.

The median PFS and OS in patients with FGFR3 mutations (n=74) and FGFR2/3 fusions (n=25) were:

Median OS (95% CI): 13.80 months (10.71; NE) vs. 10.32 months (6.97; NE)

Median PFS (95% CI): 5.59 months (4.90; 7.39) vs. 2.83 months (1.64; 5.95).

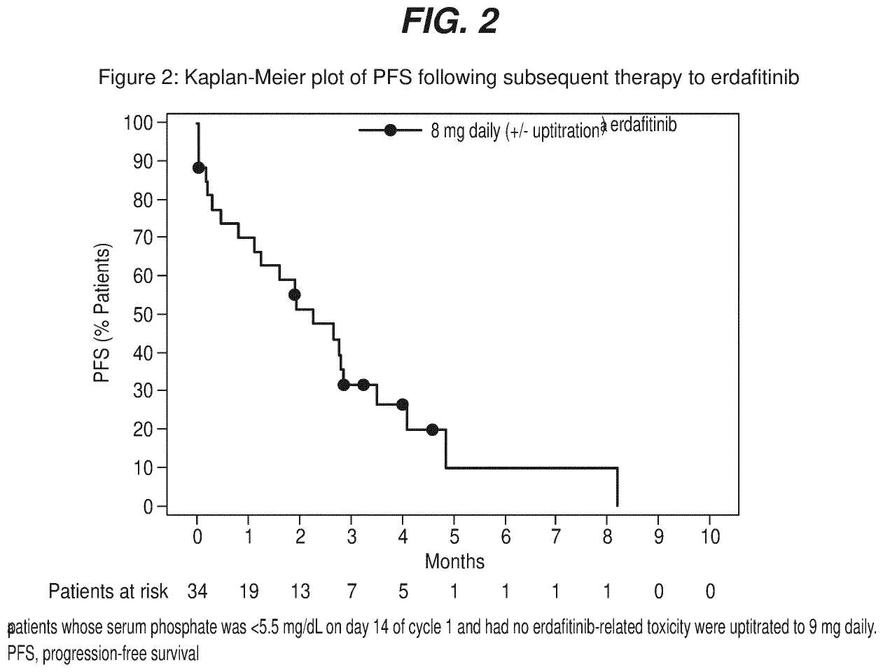

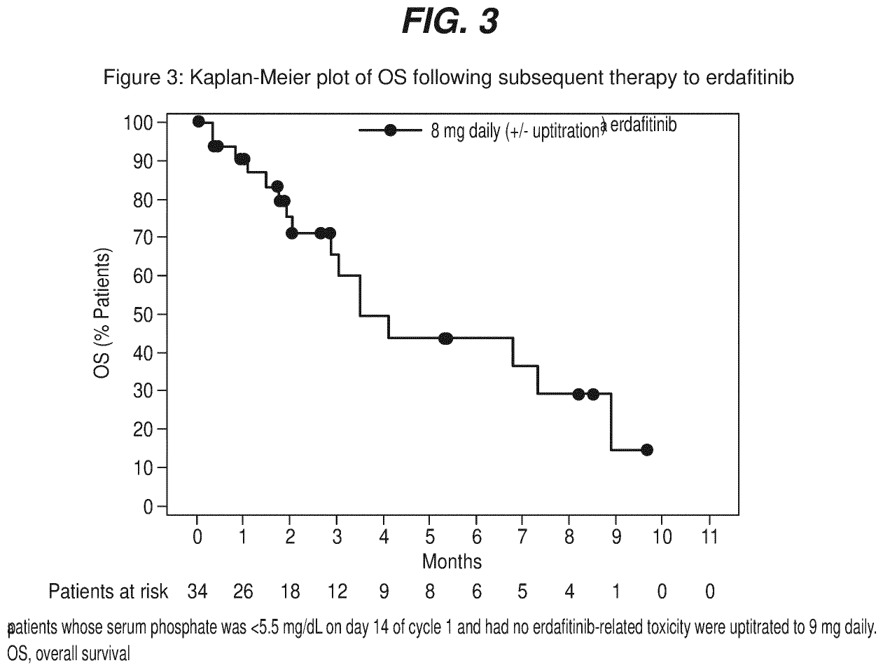

Patients who received subsequent anti-cancer therapy after erdafitinib treatment had median PFS of 2.27 months (95% CI: 0.79; 2.86) and median OS of 3.52 months (95% CI: 2.04; 8.90). See FIGS. 2 and 3.

The absence of an objective response rate (ORR) seen in the prior immunotherapy cohort that were exposed to FDA-approved I/O therapy (n=22) in BLC-2001 with an ORR in third-line immunotherapy treatment provides clinical support that erdafitinib monotherapy may have had an immune priming effect on the tumor microenvironment and highlights the potential clinical benefit of a sequential approach with checkpoint inhibitors (e.g., subsequent administration of an immune checkpoint inhibitor following monotherapy with an FGFR inhibitor such as erdafitinib).

### Example 2. Clinical Study

BLC2002 (NCT03473743) is a Phase 1b-2 Study to Evaluate Safety, Efficacy, Pharmacokinetics, and Pharmacodynamics of Erdafitinib plus Cetrelimab, an Anti-PD-1 Monoclonal Antibody, in Subjects with Metastatic or Locally Advanced Urothelial Cancer with Selected FGFR Gene Alterations.

Phase 1b is the dose escalation part of the study wherein two dosing cohorts (Standard Cohorts and Alternative Cohorts) of erdafitinib were explored, while cetrelimab intravenous (IV) dose was fixed. In the Standard Cohorts (DL1, DL2 or DL2A), erdafitinib and cetrelimab start concurrently from Cycle 1 Day 1 (C1D1). In the Alternative Cohorts (DL1B or DL2B), administration of erdafitinib starts on C1D1 but cetrelimab is initiated 1 cycle (4 weeks) later, on Cycle 2 Day 1 (C2D1) (also referred to as a 28-day run-in of erdafitinib).

Blood for immune cell profiling were collected at four time points (C1D1, C1D15, C2D1 and C3D1) and subjected to flow cytometry analysis on a real time basis. T cell activation is quantified as fold increase in the proportions of 1) CD38+CD3, CD38+CD4, or CD38+CD8 T cells out of lymphocytes or CD3 T cell populations, and 2) CD38+ cells out of CD4+ or CD8+T cell populations, compared to the baseline proportion levels at C1D1.

Longitudinal blood samples were analyzed from DL2A (E8J cohort: Erda 8 mg+cetrelimab 240 mg), DL2B (E8RJ cohort: Erda 8 mg 28 days run-in +cetrelimab 240 mg), and DL2 (EJ cohort: Erda 8 mg with the potential dose adjustments to 9 mg depending on phosphate levels measured on C1D15+cetrelimab 240 mg).

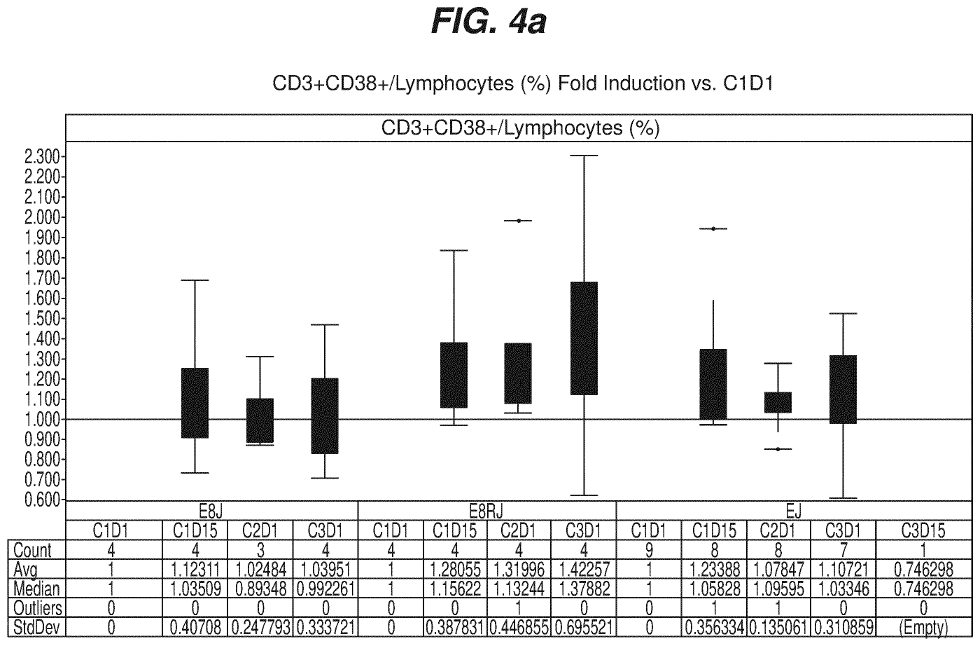

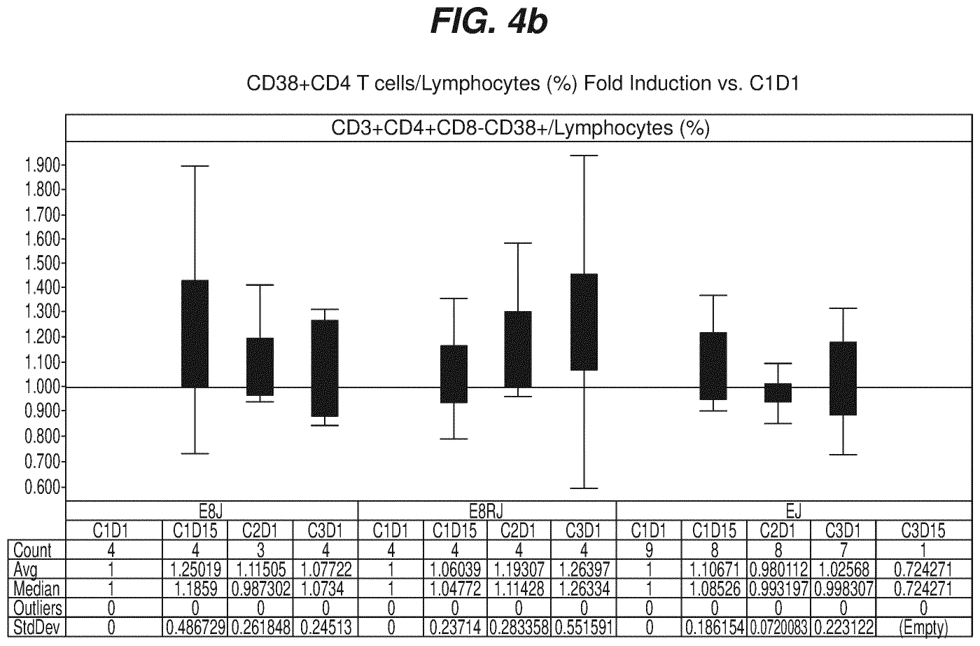

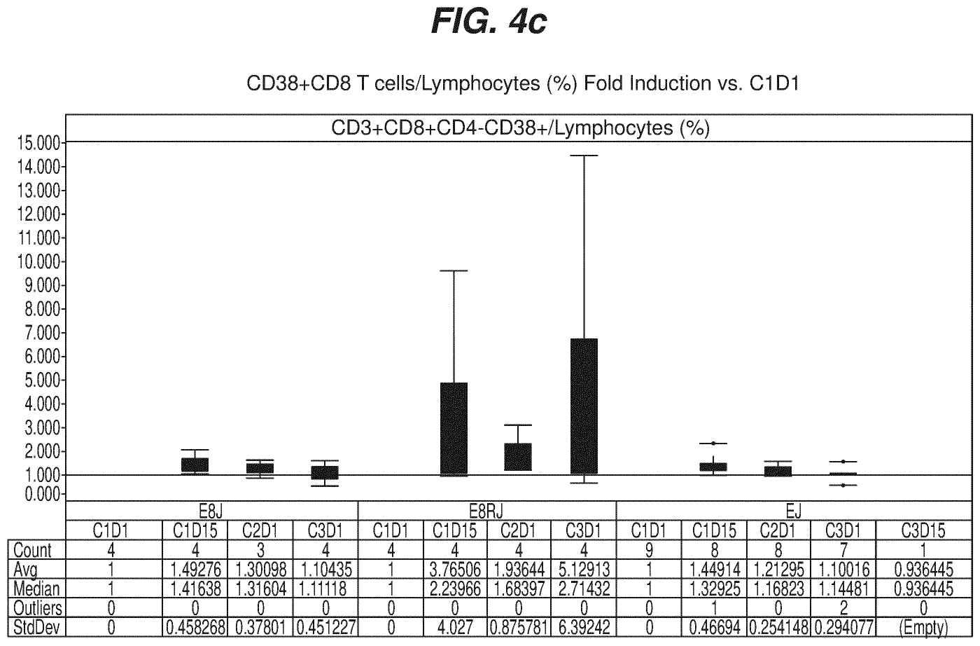

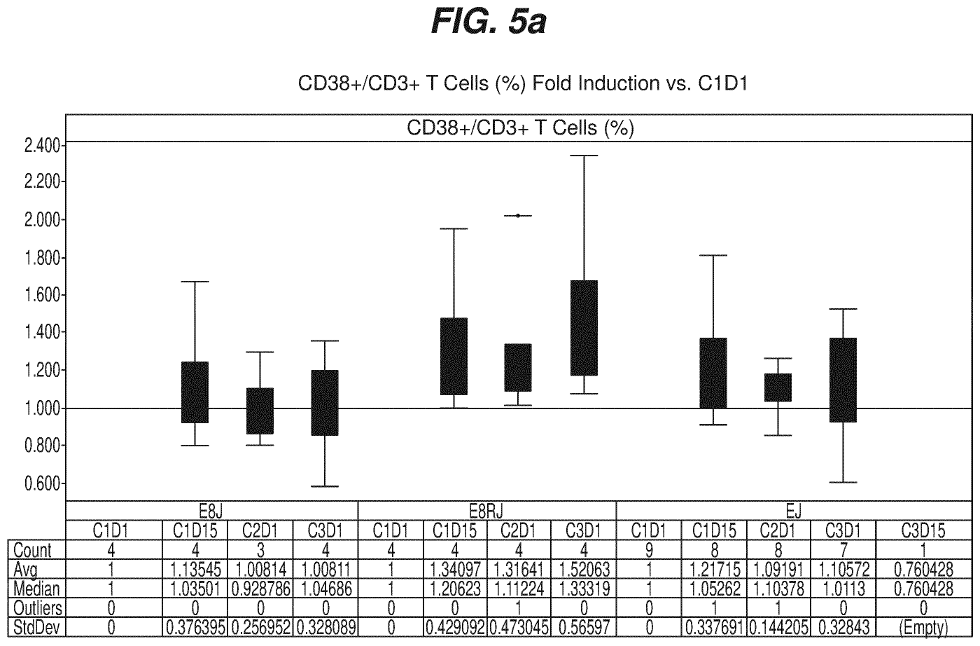

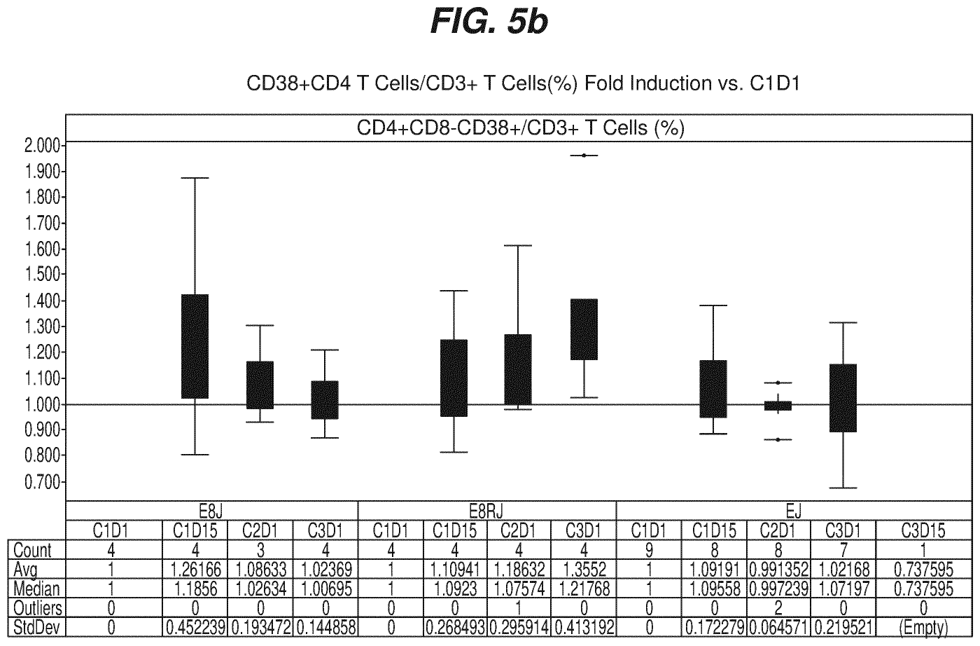

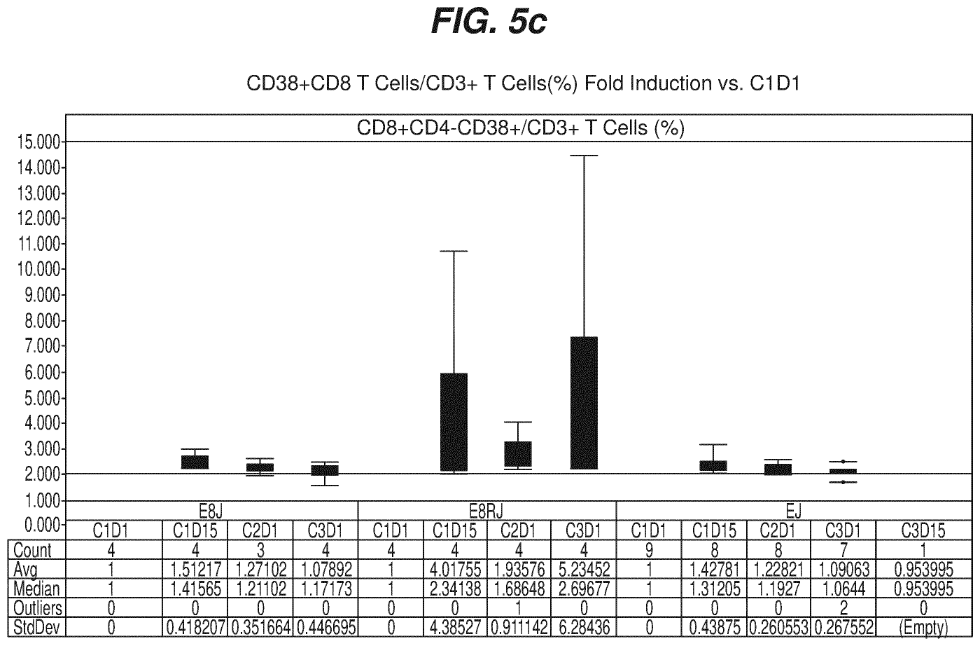

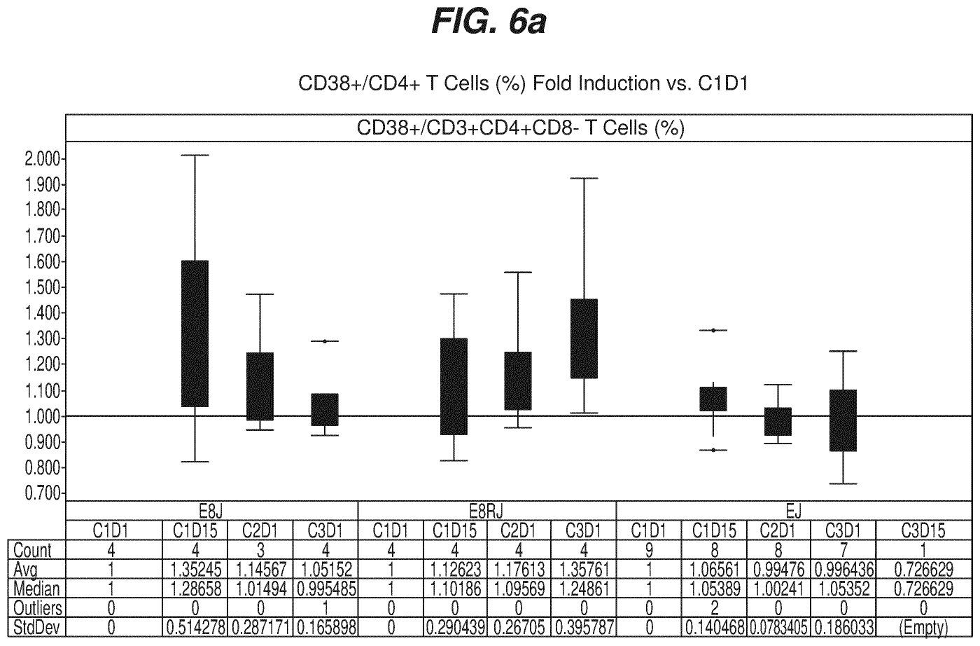

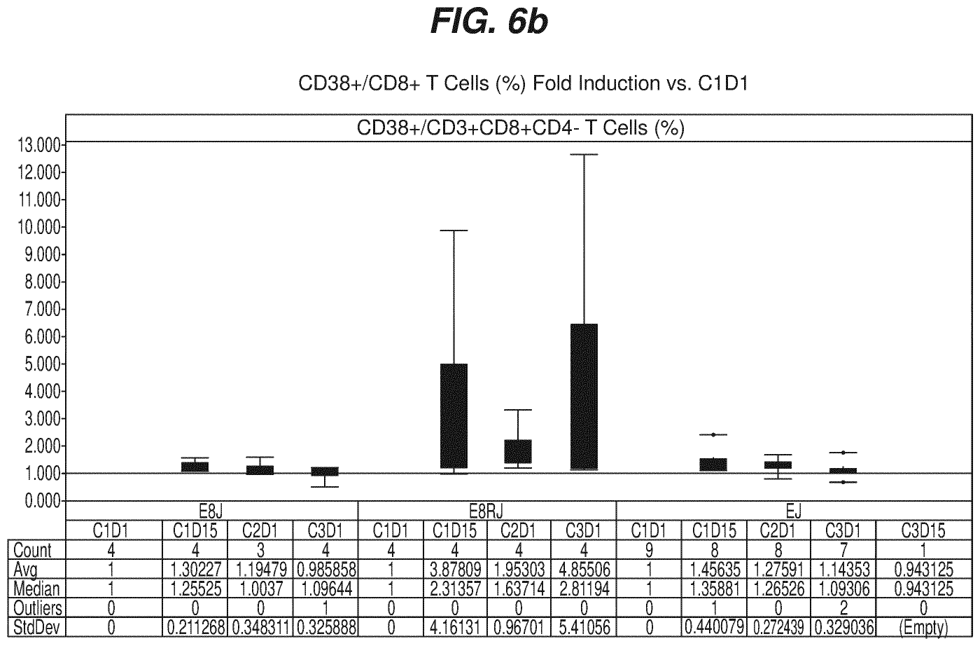

For E8RJ cohort, a sustained increase was detected in the proportion of CD38+CD3 T cell (FIG. 4a) and CD38+CD4 T cell subset (FIG. 4b) in the lymphocytes population. Interestingly, the proportion of CD38+CD8 T cells for E8RJ dramatically increased at C1D15 in response to erdafitinib treatment alone, and was further increased at C3D1 when cetrelimab was applied at C2D1 (FIG. 4c). Similar findings were observed in the proportion of CD38+CD3 T cell (FIG. 5a), CD38+CD4 T cell subset (FIG. 5b), and CD38+CD8 T cell subset (FIG. 5c) out of CD3 T cell population, as well as in the proportion of CD38+ cells out of CD4+(FIG. 6a) and CD8+ cells out of CD8+(FIG. 6b) T cell population. In contrast, the proportion of CD38+T cells for E8J and EJ only showed peak increase at C1D15 and then dropped down to the level similar to that of C1D1 at later time points. These findings indicate that sequential administration of erdafitinib followed by cetrelimab could boost and prolong the T cell activation in the peripheral blood.

1. A method of treating cancer in a patient, the method comprising: administering a therapeutically effective amount of an immune checkpoint inhibitor to the patient, wherein the patient has an FGFR variant, and has been treated with an FGFR inhibitor.

2. The method according to claim 1, wherein the FGFR inhibitor is erdafitinib or a pharmaceutically acceptable salt thereof.

3. The method according to claim 1, wherein prior to the step of administering the immune checkpoint inhibitor, the patient exhibited disease progression in response to the FGFR inhibitor.

4. The method according to claim 1, wherein prior to the step of administering the FGFR inhibitor, the patient was treated with a first immune checkpoint inhibitor and exhibited disease progression in response to said first immune checkpoint inhibitor.

5. The method according to claim 1, wherein the immune checkpoint inhibitor is an antibody that blocks the interaction between PD-1 and PD-L1.

6. The method according to claim 1, wherein the immune checkpoint inhibitor is pembrolizumab, atezolizumab or nivolumab.

7. The method according to claim 1, wherein the immune checkpoint inhibitor is an antibody that blocks the interaction between CTLA-4 and CD80 or CD86.

8. The method according to claim 1, wherein the immune checkpoint inhibitor is selected from the group consisting of atezolizumab, pembrolizumab, nivolumab, durvalumab, avelumab, anti-CSF1R antibody, tremelimumab and ipilimumab.

9. The method according to claim 1, wherein the patient has been diagnosed with an FGFR-genetically altered tumor.

10. The method according to claim 1, wherein the patient has been diagnosed with bladder cancer.

11. The method according to claim 1, wherein the patient has been diagnosed with locally advanced or metastatic urothelial cancer.

12. The method according to claim 1, wherein the patient has been diagnosed with locally advanced or metastatic urothelial cancer harboring FGFR2 or FGFR3 genetic alterations.

13. The method according to claim 1, wherein the patient has been diagnosed with locally advanced or metastatic urothelial cancer harboring FGFR2 or FGFR3 genetic alterations and who progressed during or following at least one line of prior platinum-containing chemotherapy including within 12 months of neoadjuvant or adjuvant platinum-containing chemotherapy.

14. The method according to claim 1, wherein the FGFR variant is selected from the group consisting of FGFR2:AFF3; FGFR2:BICC1; FGFR2:CASP7; FGFR2:CCDC6; FGFR2:OFD1; FGFR3:BAIAP2L1; FGFR3:TACC3-Intron; FGFR3:TACC3V1; FGFR3:TACC3V3; and a combination thereof.

15. The method according to claim 1, wherein the FGFR inhibitor is erdafitinib and is administered in an amount of between about 8 mg and about 9 mg daily.

16. The method according to claim 1, wherein the method is effective in achieving a complete or partial response in the patient.

17. The method according to claim 1, wherein the immune checkpoint inhibitor is cetrelimab.

18. A method of treating a patient diagnosed with cancer by administering an FGFR inhibitor in an amount effective to sensitize the patient to an immune checkpoint inhibitor, the method comprising:
administering an FGFR inhibitor to the patient during a second period of time, wherein an immune checkpoint inhibitor is not administered during said second period of time; and
after said second period of time, administering an immune checkpoint inhibitor to the patient during a third period of time, wherein an FGFR inhibitor is not administered to the patient during said third period of time, wherein the patient:
(a) has been diagnosed with an FGFR-genetically altered cancer,
(b) was administered a first immune checkpoint inhibitor during a first period of time prior to the second period of time, wherein an FGFR inhibitor was not administered to the patient during said first period of time, and
(c) did not respond to the first immune checkpoint inhibitor during said first period of time.

19. The method according to claim 18, wherein the FGFR inhibitor is erdafitinib or a pharmaceutically acceptable salt thereof.

20. The method according to claim 18, wherein the FGFR inhibitor is erdafitinib free base.

21. The method according to claim 18, wherein the patient responded to the FGFR inhibitor during said second period of time.

22. The method according to claim 18, wherein the immune checkpoint inhibitor is an antibody that blocks the interaction between PD-1 and PD-L1.

23. The method according to claim 18, wherein the immune checkpoint inhibitor is pembrolizumab, atezolizumab or nivolumab.

24. The method according to claim 18, wherein the immune checkpoint inhibitor is an antibody that blocks the interaction between CTLA-4 and CD80 or CD86.

25. The method according to claim 18, wherein the immune checkpoint inhibitor is selected from the group consisting of atezolizumab, pembrolizumab, nivolumab, durvalumab, avelumab, anti-CSF1R antibody, tremelimumab and ipilimumab.

26. The method according to claim 18, wherein the immune checkpoint inhibitor is cetrelimab.

27. The method according to claim 18, wherein the patient has been diagnosed with bladder cancer

28. The method according to claim 18, wherein the patient has been diagnosed with locally advanced or metastatic urothelial cancer.

29. The method according to claim 18, wherein the patient has been diagnosed with locally advanced or metastatic urothelial cancer harboring FGFR2 or FGFR3 genetic alterations.

30. The method according to claim 18, wherein the patient has been diagnosed with locally advanced or metastatic urothelial cancer harboring FGFR2 or FGFR3 genetic alterations and who progressed during or following at least one line of prior platinum-containing chemotherapy including within 12 months of neoadjuvant or adjuvant platinum-containing chemotherapy.

31. The method according to claim 18, wherein the patient has an FGFR variant selected from the group consisting of FGFR2:AFF3; FGFR2:BICC1; FGFR2:CASP7; FGFR2:CCDC6; FGFR2:OFD1; FGFR3:BAIAP2L1; FGFR3:TACC3-Intron; FGFR3:TACC3v1; FGFR3:TACC3v3; and a combination thereof.

32. The method according to claim 18, wherein the FGFR inhibitor is erdafitinib and is administered in an amount of between about 8 mg and about 9 mg daily during said second period of time.

33. The method according to claim 18, wherein the method is effective in achieving a complete or partial response in the patient, e.g., in reducing a tumor volume in the patient and/or stopping or reducing disease progression.

34-66. (canceled)
